(19) United States
(12) Patent Application Publication
(10) Pub. No.: US 2006/0173078 A1
(43) Pub. Date: Aug. 3, 2006

(54) Method of treatment or prophylaxis of symptoms of herpes viral infection

(76) Inventor: Neil McGregor, Victoria (AU)

Correspondence Address: SUGHRUE MION, PLLC, 2100 PENNSYLVANIA AVENUE, N.W., SUITE 800, WASHINGTON, DC, 20037, US

(21) Appl. No.: 10/541,794

(22) PCT Filed: Jan. 9, 2004
(86) PCT No.: PCT/AU04/00018

§ 371 Date: Mar. 2, 2006

(30) Foreign Application Priority Data
Jan. 9, 2003 (AU) 2003900064

(51) Int. Cl. A61K 31/198; A61K 31/19

(52) U.S. Cl. 514/561; 514/574

(57) ABSTRACT

A method providing the treating or prophylaxis of one or more symptoms of viral infection. The method involving the administration of citrate and/or succinate salts. The present invention also encompasses the use of citrate and/or succinate salts for the treatment of prevention of lesions or blisters, or other symptoms of infection by members of the herpes virus family.

### FIELD OF THE INVENTION

The present invention relates generally to a method for the treatment or prophylaxis of one or more symptoms of viral infection, and more particularly of herpes viral infection, in a subject. The present invention also encompasses the use of a composition for the treatment or prevention of lesions or blisters, or other symptoms of infection by members of the herpes virus family.

### BACKGROUND OF THE INVENTION

Reference to any prior art in this specification is not, and should not be taken as an acknowledgment or any form of suggestion that this prior art is common general knowledge or forms a part of the common general knowledge in Australia or any other country.

Throughout this specification, unless the context requires otherwise the word “comprise”, and variations such as “comprises” and “comprising”, will be understood to imply the inclusion of a stated element or integer or step or group of elements or integers or steps but not the exclusion of any other element or integer or step or group of elements or integers or steps.

Herpes simplex is a common viral infection characterised by the development of small fluid-filled, and initially virus-filled, blisters or sores. The infection is contagious and is spread by direct contact with blisters or the fluid they contain.

The herpes simplex virus exists in two forms known as herpes simplex virus type 1 and 2 (HSV1 and HSV2). HSV1 is usually responsible for herpes infections of the lips, mouth and face, while HSV2 is more usually, but not exclusively, associated with genital infections. It is, however, now known that either virus type can cause herpes blisters or lesions at either location. Infection occurs when a moist broken surface comes in contact with the virus, which enters the body, finds the nearest nerve, and migrates up the nerve to its root near the spinal cord where it lives for the life of the infected person.

Most adults have been exposed to HSV1 infection. The initial infection may be symptomless or may cause flu-like symptoms. In a proportion of Individuals, the virus which remains latent in the ganglia of nerve cells is periodically reactivated and causes infection in nerve cells, together with the development of skin lesions or blisters around the lips, mouth and face which are commonly known as “cold sores”. Recurrence, which is always at or around the same spot, is most likely when resistance is lowered by other illness, stress or local tissue damage, in particular recurrence is likely if the mouth is sunburned or chapped, and during respiratory infections (hence the name “cold sores”). Warning of recurrence is an itching, tingling sensation in the area. The blisters appear the following day. The infection can be passed on to another person from the time of first warning until the lesion heals. If the sufferer is or becomes immunocompromised or immunosuppressed, the infection may become a generalised infection which is potentially fatal. If infection spreads to the eyes, viral conjunctivitis or corneal ulcers can develop.

Treatment of herpes viral infection usually involves the application of the antiviral drug acyclovir which is a nucleoside analog. Secondary bacterial infections are common and may be treated with antibiotic drugs. Not all herpes viral infections are susceptible to acyclovir and furthermore, treatment is generally ineffective if it is delayed beyond the early stage of infection and symptom development. Accordingly, there is clearly a need for a new approach to the treatment or prophylaxis of herpes viral infections.

### SUMMARY OF THE INVENTION

In one aspect, the present invention provides a method for the treatment or prophylaxis of one or more symptoms of a herpes viral infection in a subject, said method comprising administration to said subject of a composition comprising a citrate salt and/or a succinate salt for a time and under conditions sufficient to treat or prevent the development of one or more symptoms of a herpes viral infection.

Still another aspect of the present invention provides a method for the treatment or prophylaxis of one or more symptoms of a herpes viral infection in a subject, said method comprising administration to said subject of a composition comprising a citrate salt and a succinate salt for a time and under conditions sufficient to treat or prevent the development of one or more symptoms of a herpes viral infection.

Yet another aspect of the present invention provides a method for the treatment or prophylaxis of one or more symptoms of a herpes viral infection in a subject, said method comprising administration to said subject of a composition comprising a citrate salt and/or a succinate salt, and at least one amino acid selected from the group comprising valine, aspartic acid, leucine, isoleucine, alanine, lysine, taurine and asparagine, for a time and under conditions sufficient to treat or prevent the development of one or more symptoms of a herpes viral infection.

Yet another related aspect of the present invention provides a composition for use in the treatment or prophylaxis of one or more symptoms of a herpes viral infection in a subject, said composition comprising a citrate salt and/or a succinate salt, and optionally at least one amino acid selected from the group comprising valine, aspartic acid, leucine, isoleucine, alanine, lysine, taurine and asparagine.

Still another aspect of the present invention provides the use of a citrate salt and/or a succinate salt, and optionally at least one amino acid selected from the group comprising valine, aspartic acid, leucine, isoleucine, alanine, lysine, taurine and asparagine, in the manufacture of a medicament for the treatment or prophylaxis of one or more symptoms of a herpes viral infection in a subject.

### DETAILED DESCRIPTION OF THE INVENTION

The present invention is predicated in part on the development of a composition for use in the treatment or prophylaxis of one or more symptoms of herpes viral infections in a subject.

In a first aspect, the present invention provides a method for the treatment or prophylaxis of one or more symptoms of a herpes viral infection in a subject, said method comprising administration to said subject of a composition comprising a citrate salt and/or a succinate salt for a time and under conditions sufficient to treat or prevent the development of one or more symptoms of a herpes viral infection.

Reference herein to the phrase “herpes viral infection” should be read as including reference to infection by any member of the herpes virus family. The herpes virus family includes herpes simplex (HSV1 and HSV2) and herpes zoster (the causative agent of shingles). In a preferred embodiment of the present invention the herpes viral infection, is a herpes simplex infection.

Reference herein to the term “prophylaxis” or “prevention” should be read as including reference to prevention or reduction of the risk of occurrence of a symptom of herpes viral infection and to prevention or reduction of the likelihood of the recurrence of a symptom of herpes viral infection.

Reference herein to the symptoms of herpes viral infection should be read as a reference to any one or more of the symptoms of herpes viral infection including a burning or tingling sensation or pain prior to the development of a lesion or blister, a local and/or general inflammatory response, or the formation and progression of blisters or lesions; and neuralgia.

In one particular embodiment, said symptom is associated with an active herpes simplex infection, either in the form of a skin lesion or blister (“cold sore”) around the lips, mouth or face, or as a genital lesion or blister (“genital sore”).

While not wishing to limit the present invention in any way to one particular theory or mode of action, it is proposed herein that the present composition is effective in inhibiting or at least reducing viral replication which is associated with the development of lesions and blisters in a herpes viral infection.

Reference herein to the term “citrate salt” includes reference to any citrate salt which is effective in reducing or enhancing the reduction of at least one of the symptoms of herpes viral infection and/or the risk of developing one or more of said symptoms.

Preferably, said citrate salt is selected from the group comprising sodium citrate, potassium citrate or magnesium citrate and the like. The choice of the cation-will depend on factors such as the mode of administration or the solubility of the citrate salt and whether or not it is desirable to include a particular cation. For example, high levels of potassium in oral formulations may affect kidney function.

Reference herein to the term “succinate salt” includes reference to any succinate salt which is effective in reducing or enhancing the reduction of at least one of the symptoms of herpes viral infection and/or the risk of developing one or more of said symptoms.

Preferably, said succinate salt is selected from the group comprising sodium succinate, potassium succinate, calcium succinate or magnesium succinate and the like. The choice of the cation will depend on factors such as the solubility of the succinate salt and whether or not it is desirable to include a particular cation. For example when considering oral formulations, high levels of potassium may affect kidney function.

In another aspect, the present invention provides a method for the treatment or prophylaxis of one or more symptoms of a herpes viral infection in a subject, said method comprising administration to said subject of a composition comprising a citrate salt and a succinate salt for a time and under conditions sufficient to treat or prevent the development of one or more symptoms of a herpes viral infection.

In one preferred embodiment of the invention, method of treatment or prophylaxis is further enhanced by including in the composition one or more amino acids selected from the group comprising; valine, aspartic acid, leucine, isoleucine, alanine, lysine, taurine and asparagine.

According to a further embodiment therefore, the present invention provides a method for the treatment or prophylaxis of one or more symptoms of a herpes viral infection in a subject, said method comprising administration to said subject of a composition comprising a citrate salt and/or succinate salt, and at least one amino acid selected from the group comprising valine, aspartic acid, leucine, isoleucine, alanine, lysine, taurine and asparagine, for a time and under conditions sufficient to treat or prevent the development of one or more symptoms of a herpes viral infection,

Reference herein to “amino acid” includes reference to derivatives, homologues, analogues and mimetics thereof which will be well known to those skilled in the art. Taurine, for example, is an analogue of b-alanine. Chemical analogues of the subject amino acids contemplated herein include, but are not limited to, modification to side chains such as amino or carboxyl groups.

In yet another aspect the present invention provides a method for the treatment or prophylaxis of one or more symptoms of a herpes viral infection in a subject, said method comprising administration to said subject of an effective amount of a citrate salt and/or a succinate salt, and optionally at least one amino acid selected from the group comprising valine, aspartic acid, leucine, isoleucine, alanine, lysine, taurine and asparagine, for a time and under conditions sufficient to treat or prevent the development of one or more symptoms of a herpes viral infection.

As previously described, the methods of the present invention are applicable particularly where the herpes infection is a herpes simplex infection, and one of the symptoms of infection is a lesion or blister (“cold sore”) around the lips, mouth or face, or a genital lesion or blister.

Another embodiment of the present invention provides a composition suitable for use in the treatment or prophylaxis of one or more symptoms of a herpes viral infection in a subject, said composition comprising a citrate salt and/or a succinate salt, and optionally at least one amino acid selected from the group comprising valine, aspartic acid, leucine, isoleucine, alanine, lysine, taurine and asparagine

Still another embodiment of the present invention provides a composition when used in the treatment or prophylaxis of one or more symptoms of a herpes viral infection in a subject, said composition comprising a citrate salt and/or a succinate salt, and optionally at least one amino acid selected from the group comprising valine, aspartic acid, leucine, isoleucine, alanine, lysine, taurine and asparagine.

In yet another embodiment, the present provides the use of a citrate salt and/or a succinate salt, and optionally at least one amino acid selected from the group comprising valine, aspartic acid, leucine, isoleucine, alanine, lysine, taurine and asparagine, in the manufacture of a medicament for the treatment or prophylaxis of one or more symptoms of a herpes viral infection in a subject.

Components of the composition may be obtained from any convenient source. For example, they may be in purified form or they maybe in the form of herbs or preferably an extract of herbs or horticultural or botanical equivalents of herbs or chemical or functional equivalents of the herb extract.

Oral administration of the composition of the present invention is contemplated although delivery may be by any convenient means such as intravenous, intranasal, intraperitoneal, subcutaneous, intradermal, topical, suppository routes or implantation (slow-release molecules).

Pharmaceutical forms of the composition may be suitable for injectable use such as sterile aqueous solutions and sterile powders for the extemporaneous preparation of sterile injectable solutions or dispersions.

The composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof and vegetable oils. The proper fluidity can be maintained, for example, by the use of a coating such as lecithin. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The compositions may be orally administered, for example, with an inert diluent or with an assimilable edible carrier, or it may be enclosed in hard or soft shell gelatin capsule, or it may be compressed into tablets, or it may be in powdered form or incorporated directly with the food of the diet. For oral therapeutic and/or prophylactic administration, the active compound may be incorporated with excipients and used in the form of ingestible tablets, buccal tablets, troches, capsules, elixirs, suspensions, syrups, wafers, and the like.

A broad range of doses may be applicable depending on the subject, severity of condition and proposed route and medium for administration. The amount of active compound in such therapeutically useful compositions is such that a suitable dosage will be obtained. Preferred compositions according to the present invention are prepared so that an oral dosage unit form contains between about 0.01 μg and about 2000 mg of active compound. Alternative amounts include between about 1.0 μg and about 1500 mg, between about 1 μg and about 1000 mg and between about 10 μg and about 500 mg.

The tablets, troches, pills, capsules and the like may also contain the components as listed hereafter: A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active Ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient or ingredients are compounded for convenient and effective administration in effective amounts with a suitable pharmaceutically acceptable carrier in dosage unit form. A unit dosage form can, for example, contain the principal active compounds in amounts ranging from 0.01 μg to about 70 g/100 grams. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients. Alternatively, amounts administered may be represented in terms of amounts/kg body weight. In this case, amounts range from about 0.001 μg to about 1000 mg/kg body weight may be administered. Preferred ranges include from about 50 μg to 500 mg 1 kg body weight 500 mg/kg body weight or about 0.01 μg to about or above 0.1 μg to about 250 mg/kg body weight are contemplated by the present invention.

Prophylactic administration is clearly contemplated herein. Preferably, the composition is administered at an early phase of lesion or blister development, for example at the tingling or burning phase. Alternatively, the composition is administered when it is believed that one of the triggers for development of lesions or blisters has been experienced. For example, triggers for development of cold sores may be sunlight, stress or a cold or other respiratory infection. The dose and frequency of dosing is determined by a number of factors including body weight, severity and location of lesions or blisters, and frequency of recurrence of lesions or blisters.

The present invention is now further described with reference to the following non-limiting Examples.

### EXAMPLE 1

The following composition is tested in subjects:

| Compound | mg/gram | |
|---|---|---|
| Sodium Citrate | 488.8 | mg |
| L-Isoleucine | 39.0 | mg |
| L-Valine | 39.0 | mg |
| L-Asparagine | 39.0 | mg |
| L-Alanine | 26.0 | mg |
| L-Leucine | 26.0 | mg |
| L-Glycine | 26.0 | mg |
| L-Serine | 26.0 | mg |
| L-Threonine | 26.0 | mg |
| L-Tyrosine | 19.5 | mg |
| L-Phenylalanine | 19.5 | mg |
| L-Glutamine | 19.5 | mg |
| L-Proline | 19.5 | mg |
| L-Tryptophan | 19.5 | mg |
| L-Lysine | 19.5 | mg |
| Taurine | 19.5 | mg |
| L-Methionine | 13.0 | mg |
| L-Arginine | 13.0 | mg |
| L-Histidine | 13.0 | mg |
| L-Cystine | 13.0 | mg |
| Magnesium aspartate | 39.0 | mg |
| Calcium succinate | 13.0 | mg |
| Nicotinamide | 7.8 | mg |
| d-alpha Tocopheryl acetate | 5.2 | mg |
| Ferrous fumarate | 5.2 | mg |
| Alpha-Lipoic acid | 2.6 | mg |
| Calcium Pantothenate | 1.3 | mg |
| Riboflavine | 0.8 | mg |
| Thiamine | 0.5 | mg |
| Betacarotene | 0.2 | mg |
| Biotin | 10 | mcg |
| Cholecalciferol | 10 | mcg |
| Cyanocobalamin | 10 | mcg |

Subjects who suffer from cold sores are administered the composition described above in the form of 300 mg capsules, and the rate at which their cold sores heal relative to a control subject is determined.

### EXAMPLE 2

The following composition is tested in subjects:

| Compound | mg/gram | |
|---|---|---|
| Sodium Citrate | 488.8 | mg |
| L-Isoleucine | 39.0 | mg |
| L-Valine | 39.0 | mg |
| L-Asparagine | 39.0 | mg |
| L-Alanine | 26.0 | mg |
| L-Leucine | 26.0 | mg |
| L-Glycine | 26.0 | mg |
| L-Serine | 26.0 | mg |
| L-Threonine | 26.0 | mg |
| L-Tyrosine | 19.5 | mg |
| L-Phenylalanine | 19.5 | mg |
| L-Glutamine | 19.5 | mg |
| L-Proline | 19.5 | mg |
| L-Tryptophan | 19.5 | mg |
| L-Lysine | 19.5 | mg |
| Taurine | 19.5 | mg |
| L-Histidine | 13.0 | mg |
| L-Cystine | 13.0 | mg |
| Magnesium aspartate | 39.0 | mg |
| Calcium succinate | 13.0 | mg |
| Nicotinamide | 7.8 | mg |
| d-alpha Tocopheryl acetate | 5.2 | mg |
| Ferrous fumarate | 5.2 | mg |
| Alpha-Lipoic acid | 2.6 | mg |
| Calcium Pantothenate | 1.3 | mg |
| Riboflavine | 0.8 | mg |
| Thiamine | 0.5 | mg |
| Betacarotene | 0.2 | mg |
| Biotin | 10 | mcg |
| Cholecalciferol | 10 | mcg |
| Cyanocobalamin | 10 | mcg |

Subjects who suffer from cold sores are administered the composition described above in the form of 300 mg capsules, and the rate at which their cold sores heal relative to a control subject is determined.

### EXAMPLE 3

A 54 year old female subject had repeated cold sores (herpes labialis) all her life as far as she can remember. The herpes lesions predominantly occurred on the right upper lip at or near the vermilion border and at the lower left lip both approximately at the junction of the areas if the lip was divided into three sections. The lesions recurred with a frequency of approximately twice per month and were usually triggered by excess sunlight, cold or with wind burn. The subject had a medical history of inflammatory bowel disease which was being treated with sialazapyrine, however the treatment was unrelated to her history of herpes recurrence and did not seem to alter the recurrence rate.

The subject took the composition of Example 1 in 300 mg capsules and has continued to do so at the time of recurrence of her herpes lesions for a period of over nine months. The first time she took the composition the herpes lesions stopped developing and healed very rapidly. The subject states that the composition is best taken at the prodromal stage at which time the lesions can be prevented from developing. She also states that if the composition is taken after the development of the lesions, they rapidly become pain-free and then heal very rapidly (approximately over a 1-2 day period). She also states that the frequency of recurrence is now less than once every 6-8 weeks, indicating both a change in lesion development and a degree of prevention of recurrence.

### EXAMPLE 4

A 27 year old male subject had frequent repeat genital herpes lesions on his genitalia since he contracted the infection at the age of 20 years of age. No significant medical history was reported. The subject reported that taking the composition of Example 1 in 300 mg capsules, resulted in the rapid healing of the herpes lesions. He states that taking the composition at the prodromal stage can prevent lesion development, and if the lesions have developed, the lesions become painless and heal rapidly. Following use of the composition for 34 months, the subject has also reported a drop in the number of recurrences of lesion development.

### EXAMPLE 5

A 32 year old female had frequent herpes labialis (cold sores) which occurred on the lower right lip at or near the vermillion border. The lesions recurred several times per year and were usually triggered by sunlight, cold or with wind burn. The female subject took the composition set forth in Example 1 on day 2 of her lesion development and reported that the cold sores stopped developing and healed very rapidly. Observation of the lesion showed that it had stopped developing and that whilst a scab developed it healed within days. The female states that the formula is best treatment that she has ever had for her herpes labialis.

### EXAMPLE 6

Observation of the lesion healing in seven different patients did not reveal anyone that the treatment did not help. All these patients who had taken the composition set forth in Example 1 were very happy to state that it was a superior treatment to any other tried including the commercially available antiviral medications.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. A method for the treatment or prophylaxis of one or more symptoms of a herpes viral infection in a subject, said method comprising administration to said subject of a composition comprising a citrate salt and/or a succinate salt for a time and under conditions sufficient to treat or prevent the development of one or more symptoms of a herpes viral infection.

2. The method according to claim 1 wherein said composition comprises a citrate salt and a succinate salt.

3. The method according to claim 1 wherein said composition further comprises an amino acid selected from vatine, aspartic acid, leucine, isoleucine, alanine, lysine, taurine and asparagine.

4. The method according to claim 1 wherein said herpes viral infection is caused by herpes simplex or herpes zoster.

5. The method according to claim 4 wherein said herpes viral infection is herpes simplex.

6. The method according to claim 1 wherein said symptom is a blister or lesion.

7. The method according to claim 1 wherein said symptom is a cold sore or a genital sore.

8. The method according to claim 1 wherein said subject is a human subject.

9. The method according to claim 1 wherein said citrate salt is sodium citrate, potassium citrate or magnesium citrate.

10. The method according to claim 1 wherein said succinate salt is sodium succinate, potassium succinate, calcium succinate or magnesium succinate.

11. The method according to claims 1 wherein said succinate salt is calcium succinate.

12. A composition for use in the treatment or prophylaxis of one or more symptoms of a herpes viral infection in a subject, said composition comprising a citrate salt and/or a succinate salt.

13. The composition according to claim 12 wherein said composition comprises a citrate salt and a succinate salt.

14. The composition according to claim 12 wherein said composition further comprises an amino acid selected from valine, aspartic acid, leucine, isoleucine, alanine, lysine, taurine and asparagine.

15. The composition according to claim 12 wherein said herpes viral infection is caused by herpes simplex or herpes zoster.

16. The composition according to claim 15 wherein said herpes viral infection is herpes simplex.

17. The composition according to claim 12 wherein said symptom is a blister or lesion.

18. The composition according to claim 12 wherein said symptom is a cold sore or a genital sore.

19. The composition according to claim 12 wherein said subject is a human subject.

20. The composition according to claim 12 wherein said citrate salt is sodium citrate, potassium citrate or magnesium citrate.

21. The composition according to claim 12 wherein said succinate salt is sodium succinate, potassium succinate, calcium succinate or magnesium succinate.

22. The composition according to claim 12 wherein said succinate salt is calcium succinate.

23. A use of a composition comprising a citrate salt and/or a succinate salt in the manufacture of a medicament for the treatment or prophylaxis of one or more symptoms of a herpes viral infection in a subject.

24. The composition according to claim 23 wherein said composition comprises a citrate salt and a succinate salt.

25. The composition according to claim 23 wherein said composition further comprises an amino acid selected from valine, aspartic acid, leucine, isoleucine, alanine, lysine, taurine and asparagine.

26. The composition according to claim 23 wherein said herpes viral infection is caused by herpes simplex or herpes zoster.

27. The composition according to claim 26 wherein said herpes viral infection is herpes simplex.

28. The composition according to claim 23 wherein said symptom is a blister or lesion.

29. The composition according to claim 23 wherein said symptom is a cold sore or a genital sore.

30. The composition according to claim 23 wherein said subject is a human subject.

31. The composition according to claim 23 wherein said citrate salt is sodium citrate, potassium citrate or magnesium citrate.

32. The composition according to claim 23 wherein said succinate salt is sodium succinate, potassium succinate, calcium succinate or magnesium succinate.

33. The composition according to claim 23 wherein said succinate salt is calcium succinate.

34. Use of a composition comprising a citrate salt and/or a succinate salt in the treatment and/or prophylaxis of one or more symptoms of the herpes viral infection in a subject.

35. The use according to claim 34 wherein said composition comprises a citrate salt and a succinate salt.

36. The use according to claim 34 wherein said composition further comprises an amino acid selected from valine, aspartic acid, leucine, isoleucine, alanine, lysine, taurine and asparagine.

37. The use according to claim 3 wherein said herpes viral infection is caused by herpes simplex or herpes zoster.

38. The use according to claim 37 wherein said herpes viral infection is herpes simplex.

39. The use according to claim 34 wherein said symptom is a blister or lesion.

40. The use according to claim 34 wherein said symptom is a cold sore or a genital sore.

41. The use according to claim 34 wherein said subject is a human subject.

42. The use according to claim 34 wherein said citrate salt is sodium citrate, potassium citrate or magnesium citrate.

43. The use according to claim 34 wherein said succinate salt is sodium succinate, potassium succinate, calcium succinate or magnesium succinate.

44. The use according to claim 34 wherein said succinate salt is calcium succinate.
